(19) United States
(12) Patent Application Publication
(10) Pub. No.: US 2009/0130217 A1
(43) Pub. Date: May 21, 2009

(54) Cosmetic Composition Containing Nanoparticulate a-Alumina

(75) Inventors: Norbert Roesch, Gersthofen (DE); Peter Klug, Grossostheim (DE); Waltraud Simsch, Kelkheim (DE)

Correspondence Address: CLARIANT CORPORATION;INTELLECTUAL PROPERTY DEPARTMENT, 4000 MONROE ROAD, CHARLOTTE, NC, 28205, US

(73) Assignee: CLARIANT INTERNATIONAL LTD., 4132 Muttenz (CH)

(21) Appl. No.: 12/293,245

(22) PCT Filed: Mar. 12, 2007
(86) PCT No.: PCT/EP2007/002129

§ 371 Date: Sep. 16, 2008

(30) Foreign Application Priority Data
Mar. 17, 2006 (DE) 10 2006 012 319.0
Jun. 28, 2006 (DE) 10 2006 029 879.9

(51) Int. Cl. A61K 9/14; A61K 47/02

(52) U.S. Cl. 424/489; 514/769; 977/926

(57) ABSTRACT

Cosmetic or dermatological preparations which contain modified or unmodified nanocrystalline a-alumina having particle sizes of from 10 to 100 nm and d50 values of from 30 to 60 nm are described.

The present invention relates to cosmetic preparations comprising nanocrystalline corundum and/or doped and/or surface-modified corundum having particle sizes in the range from 20 to 100 nm and to the use of the nanoparticles as abrasives in cosmetic preparations.

The use of nanoparticulate compounds, of sparing water solubility, of an element of main groups 2 to 4 or of transition groups 2/4/8 of the Periodic Table for the abrasive treatment of the skin is known.

WO 99/45901 describes the use of tourmaline powder with 0.1 to 10 μm in cleansing compositions for the skin and for the hair.

EP 1249227 discloses cosmetic compositions with antiwrinkle effect comprising a mineral filler, preferably silicon compounds in the form of colloidal particles having sizes from 0.1 to 100 nm.

WO 02/051376 discloses abrasive compositions for treating skin which comprise nanoparticulate compounds having particle sizes from 10 to 100 nm, examples being zinc oxide, magnesium silicate, aluminas such as Al₂O₃ C from Degussa or aluminum oxide hydroxide (boehmite from Condea).

US 2005/0037038 describes a cosmetic dermabrasion process according to which, in the first step, unevennesses on the skin are eliminated using adhesives, in the second step the skin is smoothed with compositions which comprise solid particles, examples being siliceous earth, metal oxides, including, for example, corundum, and wax particles, clays, etc., and then a treatment takes place for regenerating the skin.

EP 336900 discloses skincare compositions comprising an abrasive, examples being crosslinked polystyrenes, polymethyl methacrylates, polyethylene, polyester fibers or aluminum oxide having particle sizes from 3 to 10 μm.

Solid particles of high hardness and sizes >1 μm, although effective abrasives, nevertheless give the formulations unpleasant sensorial qualities on the skin and can lead to instances of damage and irritation to the skin.

Very fine abrasives, on the other hand, with particle sizes <20 nm, are often difficult to handle, and, in the case of aluminum compounds, are toxic on inhalation. The capacity for them to be processed to homogeneous formulations is complicated, and the phase stability is inadequate.

It was an object of the present invention to provide abrasives for cosmetic products that have a hardness which gives them a good abrasive effect, but at the same time also exhibit good skin sensorial properties, can be effectively incorporated into formulations and dispersed homogeneously in formulations, and do not tend toward phase separation on storage.

Surprisingly it has been found that nanocrystalline corundum or nanocrystalline modified corundum, prepared from aluminum chlorohydrate or from aluminum chlorohydrate and oxides and/or oxide formers such as chlorides, oxychlorides, carbonates, nitrates, sulfates and/or hydroxychlorides of the elements Ca, Mg, Y, Ti, Zr, Cr, Fe, Co and/or Si, preferably of the elements Ca and/or Mg, in the form of a solution, which is first admixed with very finely disperse crystallization nuclei, preferably α-Al₂O₃ nuclei, and subsequently is subjected to a thermal or thermophysical reaction, and the resulting agglomerates are comminuted to particle sizes from 10 to 100 nm, preferably 20 to 100 nm, and, if desired, are surface modified, are outstandingly suitable as abrasives for cosmetic preparations.

The invention provides cosmetic or dermatological preparations comprising optionally modified nanocrystalline corundum having particle sizes from 10 to 100 nm, preferably 20 to 100 nm, and d50 values from 30 to 60 nm.

Preference is given to cosmetic or dermatological preparations comprising optionally modified nanocrystalline corundum having particle sizes from 10 to 100 nm, preferably 20 to 100 nm, and d50 values from 30 to 60 nm, characterized in that the optionally modified nanocrystalline corundum is obtainable by a process where at least
a) aluminum chlorohydrate of the formula Al₂(OH)ₓCl_{y}, in which x is a number from 2.5 to 5.5 and y is a number from 3.5 to 0.5 and the sum of x and y is always 6, in the form of an aqueous solution, preferably in the form of a 50% strength by weight aqueous solution, is mixed with solid, pulverulent crystallization nuclei having an average particle size of less than 0.1 μm, preferably selected from hematite, diaspore, and α-Al₂O₃, more preferably α-Al₂O₃, and which is preferably in the form of a suspension,
b) the product from step a) is dried and optionally comminuted,
c) the product from step b) is subjected to a thermal treatment (calcining) at 500 to 1100° C., preferably at 700 to 1100° C., more preferably at 1000 to 1100° C., more particularly for 2 to 30 minutes, and
d) the resulting agglomerates from step c) are comminuted by wet or dry grinding, preferably by wet grinding, more particularly in water.

In the context of the present invention, with regard to step d) described above, the term “deagglomeration” is also used.

Through the use of nanocrystalline corundum, more particularly nanocrystalline corundum having a hardness of 9, it is possible very effectively to remove even small skin unevennesses and small skin flakes of the stratum corneum without causing a disruptive granular, sandy sensation. Compositions comprising nanocrystalline corundum having the particle sizes stated above feature very good skin sensorial properties with a very effective skin-smoothing effect, and are superior to the conventional peeling compositions. The spherical particles having sizes from 10 to 100 nm, preferably 20 to 100 nm, have good processing properties and can be homogeneously distributed and stably dispersed effectively.

In the context of the present invention, terms such as nanocrystalline corundum, nanoparticulate corundum, and nanocorundum are used synonymously.

The thermal treatment of step c) takes place preferably in a push-through, chamber, muffle, tube, rotary-tube or microwave furnace or in a fluidized-bed reactor.

In one preferred embodiment of the invention the nanocrystalline corundum contained in the preparations of the invention is unmodified.

In another preferred embodiment of the invention the nanocrystalline corundum contained in the preparations of the invention is modified.

In one particularly preferred embodiment of the invention the preparations of the invention comprise modified nanocrystalline corundum which is obtainable by a process where the aqueous composition from step a) comprises, in addition to aluminum chlorohydrate and the crystallization nuclei, at least one oxide and/or one oxide former.

Using at least one oxide and/or oxide former alongside the aluminum chlorohydrate and the crystallization nuclei in the above-described step a) produces nanocrystalline doped corundum.

Said at least one oxide is preferably selected from calcium oxide, magnesium oxide, chromium(III) oxide, Fe(II) oxide and/or Ti(IV) oxide, and more preferably from calcium oxide and magnesium oxide.

Said at least one oxide former is preferably selected from a chloride, oxychloride, carbonate, nitrate, sulfate and/or hydroxychloride, more particularly from a chloride, oxychloride, nitrate and/or hydroxychloride, of the elements of main groups II to V and also of the transition groups.

With particular preference said at least one oxide former is selected from a chloride, oxychloride, carbonate, nitrate, sulfate and/or hydroxychloride, preferably from a chloride, oxychloride and/or hydroxychloride, of the elements Ca, Mg, Y, Ti, Zr, Cr, Fe, Co, and Si, more particularly of the elements Ca and/or Mg.

The conversion to the oxide takes place for these materials, i.e., the oxide formers, in the course of calcining.

In a further preferred embodiment of the invention the modified nanocrystalline corundum is doped with calcium oxide and/or with magnesium oxide. In one particularly preferred embodiment of the invention this doped nanocrystalline corundum is not surface modified. In a further particularly preferred embodiment of the invention this doped nanocrystalline corundum is additionally surface modified.

The amount of added oxides and/or oxide formers is selected such that there are 0.01% to 5% by weight and preferably 0.05% to 2% by weight of additional oxide in the end product, i.e., in the modified nanocrystalline corundum.

In this way it is possible as well, for example, by doping with trace elements, to gain access to minerals such as sapphire (addition of iron, preferably iron(II) sulfate or iron(II) chloride, and titanium oxide formers, preferably TiCl₄ or titanium oxide sulfate) or ruby (addition of chromium oxide formers, preferably chromium(III) chloride, corresponding typically to 0.1% to 0.7% by weight Cr₂O₃) in nanoparticulate form.

As already partly remarked, the initial solution, more particularly the aqueous composition from step a), in one preferred embodiment of the invention comprises, in addition to aluminum chlorohydrate and the crystallization nuclei, one or more oxides and/or oxide formers, in order to generate doped corundum in the sense of the present invention. Particularly suitable for this purpose are the chlorides of the elements of main groups I and II of the Periodic Table of the Elements, more particularly the chlorides of the elements Ca and Mg, but also, furthermore, the oxides and other soluble or dispersible salts such as oxychlorides, carbonates, nitrates, sulfates or hydroxychlorides, and preferably oxides, oxychlorides, carbonates or sulfates. The main group I and II oxides may be present as a separate phase alongside the aluminum oxide, or may form true mixed oxides with it. The term “mixed oxides” in the context of this invention should be understood to include both types and to be embraced by the term “doped corundum” used in the context of the present invention.

In a further preferred embodiment of the invention the preparations of the invention comprise nanocrystalline corundum which is coated, i.e., surface modified, with one or more surface modifiers.

Examples of suitable surface modifiers for the nanoparticulate corundum are monobasic or polybasic carboxylic acids having 2 to 18 carbon atoms, hydroxycarboxylic acids, ethercarboxylic acids, fruit acids and amino acids.

For the surface modification of the nanoparticulate corundum it is also possible with preference to use silanes, siloxanes, functional silanes or functional siloxanes, which are referred to uniformly below as silanes.

For the surface modification of the nanoparticles with silanes there are two possibilities.

According to the first variant, the deagglomeration may be performed, in accordance with step d) described above, in the presence of the silane: for example, by feeding the silane into the mill during grinding.

A second possibility involves first disrupting the agglomerates of the nanocorundum and then treating the nanoparticles subsequently, i.e., after the above-described step d), preferably in the form of a suspension in an organic solvent, with the silane.

Preferred silanes are compounds of the formula

R[—Si(R′R″)—O—]ₙSi(R′R″)—R′″ or cyclo-[-Si(R′R″)—O—]ᵣSi(R′R″)—O—

in which
R, R′, R″, and R′″, identical or different from one another, are each an alkyl radical having 1 to 18 C atoms or a phenyl radical or an alkylphenyl or a phenylalkyl radical having 7 to 18 C atoms, or a radical of the general formula —(CₘH₂ₘ—O)ₚ—C_{q}H_{2q+1} or a radical of the general formula —CₛH₂ₛY or a radical of the general formula -XZₜ₋₁,
n is an integer, where 1≦n≦1000, preferably 1≦n≦100,
r is an integer, where 2≦r≦10,
m is an integer, where 0≦m≦12,
p is an integer, where 0≦p≦60,
q is an integer, where 0≦q≦40,
s is an integer, where 0≦s≦18,
Y is a reactive group, preferably α,β-ethylenically unsaturated groups, more preferably (meth)acryloyl, vinyl or allyl groups, amino, amido, ureido, hydroxyl, epoxy, isocyanato, mercapto, sulfonyl, phosphonyl, trialkoxysilyl, alkyldialkoxysilyl, dialkylmonoalkoxysilyl, anhydride and/or carboxyl groups, imido, imino, sulfite, sulfate, sulfonate, phosphine, phosphite, phosphate or phosphonate groups,
X is a radical of a t-functional oligomer, t being an integer and 2≦t≦8, and
Z in turn is a radical derived from compounds of the formula

R[—Si(R′R″)—O—]ₙSi(R′R″)—R′″ or cyclo-[-Si(R′R″)—O—]ᵣSi(R′R″)—O—

where the radicals R, R′, R″, R′″, and n and r are as defined above.

The t-functional oligomer X is preferably selected from: oligoether, oligoester, oligoamide, oligourethane, oligourea, oligoolefin, oligovinyl halide, oligovinylidene dihalide, oligoimine, oligovinyl alcohol, ester, acetal or ether of oligovinyl alcohol, cooligomers of maleic anhydride, oligomers of (meth)acrylic acid, oligomers of (meth)acrylic esters, oligomers of (meth)acrylamides, oligomers of (meth)acrylimides or oligomers of (meth)acrylonitrile, more preferably oligoethers, oligoesters or oligourethanes.

Preferred radicals of oligoethers are compounds of the type —(CₐH₂ₐ—O)_{b}—CₐH₂ₐ— or O—(CₐH₂ₐ—O)_{b}—Cₐ—H₂ₐ—O where a is an integer with 2≦a≦12 and b is an integer with 1≦b≦60, more particularly a diethylene glycol, triethylene glycol or tetraethylene glycol radical, a dipropylene glycol, tripropylene glycol, tetrapropylene glycol radical, a dibutylene glycol, tributylene glycol or tetrabutylene glycol radical.

Preferred radicals of oligoethers are compounds of the type —C_{b}H_{2b}—(O(CO)CₐH₂ₐ—(CO)O—C_{b}H_{2b}—)_{c} or —O—C_{b}H_{2b}—O(CO)CₐH₂ₐ—(CO)O—C_{b}H_{2b}—)_{c}—O— where a and b are integers, can be identical or different, and are subject to 3≦a≦12, 3≦b≦12, and 1≦c≦30, more particularly a radical of an oligoester of hexanediol and adipic acid.

Additionally preferred silanes are organosilanes of the formula

(RO)₃Si(CH₂)ₘ—R′

in which
R is alkyl having 1 to 6 C atoms, preferably methyl, ethyl or propyl,
m is an integer from 0 to 20, preferably 1 to 20,
R′ is methyl, phenyl,
   —C₄F₉; OCF₂—CHF—CF₃, —C₆F₁₃, —O—CF₂—CHF₂,
   —NH₂, —N₃, —SCN, —CH═CH₂, —NH—CH₂—CH₂—NH₂,
   —N—(CH₂—CH₂—NH₂)₂,
   —OOC(CH₃)C═CH₂,
   —OCH₂—CH(O)CH₂,
   —NH—CO—N—CO—(CH₂)₅,
   —NH—COO—CH₃, —NH—COO—CH₂—CH₃, —NH—(CH₂)₃Si(OR)₃,
   —Sₓ—(CH₂)₃Si(OR)₃,
   —SH,
   —NR″R′″R″″ in which R″ is alkyl having 1 to 6 C atoms or phenyl; R′″ is alkyl having 1 to 6 C atoms or phenyl; R″″ is H, alkyl having 1 to 6 C atoms, phenyl, benzyl or
   C₂H₄NR′″″R″″″, in which R′″″ is H or alkyl having 1 to 6 C atoms and R″″″ is H or alkyl having 1 to 6 C atoms.

The radical —NH—CO—N—CO—(CH₂)₅ is cyclic and corresponds to the following formula

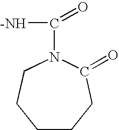

Preferred silanes of the type defined above are hexamethyldisiloxane, octamethyltrisiloxane, further homologous and isomeric compounds of the series SiₙOₙ₋₁(CH₃)₂ₙ₊₂, where n is an integer and 2≦n≦1000, e.g., polydimethylsiloxane 200® fluid (20 cST), hexamethylcyclotrisiloxane, octamethylcyclotetrasiloxane, decamethylcyclopentasiloxane, further homologous and isomeric compounds of the series (Si—O)ᵣ(CH₃)₂ᵣ, where r is an integer with 3≦r≦12, dihydroxytetramethyldisiloxane, dihydroxyhexamethyltrisiloxane, dihydroxyoctamethyltetrasiloxane, further homologous and isomeric compounds of the series HO—[(Si—O)ₙ(CH₃)₂ₙ]—Si(CH₃)₂—OH or HO—[(Si—O)ₙ(CH₃)₂ₙ]—[Si—O)ₘ(C₆H₅)₂ₘ]—Si(CH₃)₂—OH, -co-diphenylsiloxane, where n is an integer from 2 to 1000 and m is an integer with 2≦m≦1000, preferably α,ω-dihydroxypolysiloxanes, e.g., polydimethylsiloxane (OH end groups, 90-150 cST) or polydimethylsiloxane (dihydroxy end groups, 60 cST), dihydrohexamethyltrisiloxane, dihydrooctamethyltetrasiloxane, further homologous and isomeric compounds of the series H—[(Si—O)ₙ(CH₃)₂ₙ]—Si(CH₃)₂—H where n is an integer with 2≦n≦1000, preferably α,ω-dihydropolysiloxanes, e.g., polydimethylsiloxane (hydride end groups, Mₙ=580), di(hydroxypropyl)hexamethyltrisiloxane, di(hydroxypropyl)-octamethyltetrasiloxane, further homologous and isomeric compounds of the series HO—(CH₂)ᵤ[(Si—O)ₙ(CH₃)₂ₙ]—Si(CH₃)₂(CH₂)ᵤ—OH where u is an integer, with 3≦u≦18, and n is an integer, with 3≦n≦1000, preferably α,ω-dicarbinolpolysiloxanes or their polyether-modified successor compounds based on ethylene oxide (EO) and propylene oxide (PO) in the form of a homopolymer or copolymer HO-(EO/PO)ᵥ—(CH₂)ᵤ[(Si—O)ₜ(CH₃)₂ₜ]—Si(CH₃)₂(CH₂)ᵤ-(EO/PO)ᵥ—OH where t is an integer with 3≦t≦1000, u is an integer with 3≦u≦18, and v is an integer with 1≦v≦50, preferably α,ω-di(carbinol polyether)-polysiloxanes.

Likewise employed instead of α,ω-OH groups are the corresponding difunctional compounds with epoxy, isocyanato, vinyl, allyl and di(meth)-acryloyl groups, e.g., polydimethylsiloxane with vinyl end groups (850-1150 cST) or Tegorad 2500 from Tego Chemie Service.

Also suitable are the esterification products of ethoxylated/propoxylated trisiloxanes and higher siloxanes with acrylic acid copolymers and/or maleic acid copolymers as a modifying compound, e.g., BYK Silclean 3700 from Byk Chemie or Tego® Protect 5001 from Tego Chemie Service GmbH.

Likewise employed instead of α,ω-OH groups are the corresponding difunctional compounds with —NHR″″, in which R″″ is H or alkyl having 1 to 6 C atoms, e.g., the common-knowledge aminosilicone oils from the companies Wacker, Dow Corning, Bayer, Rhodia, etc., which on their polymer chain carry (cyclo)-alkylamino groups or (cyclo)-alkylimino groups distributed randomly on the polysiloxane chain.

Additionally preferred as silanes are organosilanes of the formula

(RO)₃Si(CₙH₂ₙ₊₁)

in which
R is alkyl having 1 to 26 C atoms, preferably methyl, ethyl, n-propyl, isopropyl or butyl, and
n is an integer from 1 to 26, preferably 1 to 20.

Additionally preferred as silanes are organosilanes of the formula

R′ₓ(RO)_{y}Si(CₙH₂ₙ₊₁)

in which
R is alkyl having 1 to 26 C atoms, preferably methyl, ethyl, n-propyl, isopropyl or butyl,
R′ is alkyl having 1 to 6 C atoms, preferably methyl, ethyl, n-propyl, isopropyl or butyl, or cycloalkyl having 5 to 12 C atoms,
n is an integer from 1 to 20,
x is 1 or 2,
y is 1 or 2, and
x+y is 3.

Additionally preferred silanes are the silanes listed below: triethoxysilane, octadecyltrimethoxysilane, 3-(trimethoxysilyl)propyl methacrylates, 3-(trimethoxysilyl)propyl acrylates, 3-(trimethoxysilyl)methyl methacrylates, 3-(trimethoxysilyl)methyl acrylates, 3-(trimethoxysilyl)ethyl methacrylates, 3-(trimethoxysilyl)ethyl acrylates, 3-(trimethoxysilyl)pentyl methacrylates, 3-(trimethoxysilyl)pentyl acrylates, 3-(trimethoxysilyl)hexyl methacrylates, 3-(trimethoxysilyl)hexyl acrylates, 3-(trimethoxysilyl)butyl methacrylates, 3-(trimethoxysilyl)butyl acrylates, 3-(trimethoxysilyl)heptyl methacrylates, 3-(trimethoxysilyl)heptyl acrylates, 3-(trimethoxysilyl)octyl methacrylates, 3-(trimethoxysilyl)octyl acrylates, methyltrimethoxysilanes, methyltriethoxysilanes, propyltrimethoxysilanes, propyltriethoxysilanes, isobutyltrimethoxysilanes, isobutyltriethoxysilanes, octyltrimethoxysilanes, octyltriethoxysilanes, hexadecyltrimethoxysilane, phenyltrimethoxysilanes, phenyltriethoxysilanes, tridecafluoro-1,1,2,2-tetrahydrooctyltriethoxysilanes, tetramethoxysilanes, tetraethoxysilane, oligomeric tetraethoxysilanes (Dynasil® 40 from Degussa), tetra-n-propoxysilanes, 3-glycidyloxypropyltrimethoxysilane, 3-g lycidyloxypropyltriethoxysilanes, 3-methacryloyloxypropyltrimethoxysilanes, vinyltrimethoxysilanes, vinyltriethoxysilanes, 3-mercaptopropyltrimethoxysilanes, 3-aminopropyltriethoxysilanes, 3-aminopropyltrimethoxysilanes, 2-aminoethyl-3-aminopropyltrimethoxysilanes, triamino-functional propyltrimethoxysilanes (Dynasylan® Triamino from Degussa), N-(n-butyl)-3-aminopropyltrimethoxysilanes, 3-aminopropylmethyldiethoxysilanes.

The silanes are added preferably in molar ratios of corundum to silane of 1:1 to 10:1. The amount of organic solvent in the deagglomeration is generally 80% to 90% by weight, based on the total amount of corundum and solvent. Solvents which can be used include in principle all organic solvents. Preference is given to using C₁-C₄ alcohols, more particularly methanol, ethanol or isopropanol, and also to acetone or tetrahydrofuran.

The deagglomeration by grinding and simultaneous modification with the silane takes place preferably at temperatures of 20 to 150° C., more preferably at 20 to 90° C.

Where the deagglomeration takes place by grinding, the suspension is subsequently separated from the grinding beads.

Following the deagglomeration, the suspension can be heated for up to 30 hours in order to complete the reaction. Finally the solvent is removed by distillation and the residue which remains is dried.

It is also possible to suspend the corundum in the corresponding solvents and to carry out the reaction with the silane after the deagglomeration in a further step.

Nanoparticles modified by this process contain preferably between 1% and 30% by weight, more preferably between 2% and 20% by weight, of the surface modifier, based on the total weight of the surface-modified nanoparticles.

In another preferred embodiment of the invention the unmodified or modified nanocrystalline corundum in the preparations of the invention has particle sizes of 20 to 80 nm, preferably of 40 to 70 nm, and more preferably of 50 to 60 nm.

The nanocrystalline corundum and/or nanocrystalline doped corundum and/or nanocrystalline surface-modified corundum described above and used in accordance with the invention is employed in the preparations of the invention in amounts of preferably 0.1% to 20%, more preferably 0.5% to 15%, and with particular preference 1% to 10% by weight, based on the completed preparations.

In addition to the abrasives stated above, the preparations of the invention may comprise further ingredients, such as thickeners, gellants, abrasive components, surface-active compounds, humectants, preservatives, pH modifiers, stabilizers, conditioning agents, dyes, fragrances, solvents, hydrotropes, glycols and polyols, cosmetic or dermatological actives, e.g., antimicrobial, sebosuppressive, and antiinflammatory actives, AHA acids (alpha-hydroxy acids), plant extracts, vitamins, and proteins and protein derivatives.

In another preferred embodiment of the invention preparations of the invention comprise one or more water-soluble or water-swellable, crosslinked or noncrosslinked, homopolymers or copolymers based on acryloyidimethyltaurine or salts thereof and/or one or more hydrophobically modified copolymers based on acryloyidimethyltaurine or salts thereof.

Preferred polymers based on acryloyldimethyltaurine or salts thereof are those as described in EP 1 069 142.

Particularly preferred are copolymers based on acrylamidoalkylsulfonic acids and cyclic N-vinylcarboxamides or based on acrylamidoalkylsulfonic acids and cyclic and linear N-vinylcarboxamides, such as Aristoflex® AVC (Clariant), or copolymers based on acrylamidoalkylsulfonic acids and acrylic esters of ethoxylated fatty alcohols, such as Aristoflex® HMB (Clariant).

Additionally suitable as thickeners are
polymers based on methacrylic acid or acrylic acid and modified (meth)acrylic acid, examples being crosslinked polymers of acrylic acid of the kind available under the trade names Carbopol 980, 981, 954, 2984, and 5984 (CTFA name: Carbomer) or Synthalen M and Synthalen K;
copolymers of (meth)acrylic acid and polyalkylene polyethers;
hydrophobically modified poly(meth)acrylates, examples being the copolymers available as Pemulen® TR-1 and TR-2 from BF Goodrich, Carbopol ETD 2020 from BF Goodrich (acrylate/C₁₀₋₃₀ alkyl acrylate polymer), Aculyn® 22 from Rohm and Haas (acrylates/steareth-20 methacrylate copolymer), Aculyn® 28 from Rohm and Haas (acrylates/beheneth-25 methacrylate copolymer), Synthalen W 2000 from 3V Sigma (acrylate/palmeth-25 acrylate copolymer), Structure 3001 from National Starch (acrylates/ceteth-20 itaconate copolymer);
homopolymers of dimethylaminoethyl methacrylates, quaternized with methyl chloride, as obtainable under the trade names Salcare 95 and Salcare 96;
copolymers of dimethylaminoethyl methacrylate, quaternized with methyl chloride and acrylamide, as obtainable under the trade names Salcare SC92 or PAS 5194;
crosslinked copolymers of vinyl isodecanoate and (meth)acrylic acid, as obtainable under the trade name Stabylen 30;
polyvinyl alcohols;
polyvinyl methyl ethers;
polyacrylamides;
polyvinylamides;
polyvinylpyrrolidone;
polyacrylic esters;
polyethylene oxides;
copolymers of maleic anhydride and vinyl methyl ether;
polysulfonic acids, more particularly copolymers based on acrylamidoalkylsulfonic acid or salts thereof and cyclic N-vinylcarboxamides or cyclic and linear N-vinylcarboxamides or else hydrophobically modified crosslinked acrylamido-alkylsulfonic acid copolymers; or
polyacrylic acid, polyacrylic acid derivatives, as described in DE 10059826.

The desired viscosity of the preparations of the invention can also be brought about by addition of cellulose ether and other cellulose derivatives (e.g., carboxymethylcellulose, hydroxyethylcellulose), gelatin, starch and starch derivatives, sodium alginates, fatty acid polyethylene glycol esters, agar agar, guar, plant gums, and microbial polysaccharides such as xanthan gum, tragacanth or dextrin derivatives, more particularly dextrin esters, dextrin palmitate for example, but also fatty acid soaps, fatty alcohols, and silicone waxes, examples being alkylmethicones, SilCare® 41M40, SilCare® 41M50, SilCare® 41M65, SilCare® 41M70 or SilCare® 41M80, and also inorganic substances such as, for example, phyllosilicates, e.g., bentonite, montmorillonite, kaolin, talc, organically modified phyllosilicates, aluminosilicates, and fumed silicas.

Suitable gellants are all surface-active compounds which in solution in the liquid phase form a network structure and so strengthen the liquid phase. Suitable gelling agents are disclosed for example in WO 98/58625.

Preferred gellants are metal salts of fatty acids, preferably having 12 to 22 C atoms, examples being sodium stearate, sodium palmitate, sodium laurate, sodium arachidates, sodium behenate, potassium stearate, potassium palmitate, sodium myristate, aluminum monostearate, hydroxy fatty acids, examples being 12-hydroxystearic acid, 16-hydroxyhexadecanoylic acid; fatty acid amides; fatty acid alkanolamides; dibenzalsorbitol, and alcohol-soluble polyamides and polyacrylamides or mixtures of such.

The preparations of the invention preferably contain 0.01% to 20%, more preferably 0.1% to 10%, with more particular preference 1% to 8%, and very preferably 3% to 7% by weight of gelling agents.

The preparations of the invention may comprise further abrasive components, examples being ground plant parts such as almond bran or wheat bran, the ground endocarp of apricot, peach, walnut or cherry stones, or the ground, optionally degreased fruit of almonds, coconuts, jojoba fruits, macadamia nuts, plant flours, e.g., maize cob flour, wheat bran, oat flour, wood flour, crystalline cellulose, hydrogenated jojoba oil, polymer beads, preferably of polyethylene or polyamide-11, having average diameters of 90-600 μm, and of active-containing microcapsules or milli-capsules which comprise petrochemical polymers (e.g., from polyamide such as nylon-11) and/or biopolymers such as gelatin, pectin, plant gums, alginates, and carrageenan.

Surface-active compounds employed include anionic, nonionic, cationic, and amphoteric surfactants, cosurfactants, and emulsifiers.

Anionic detersive substances preferably include: C₁₀-C₂₀ alkyl- and alkylene-carboxylates, alkyl ether carboxylates, fatty alcohol sulfates, fatty alcohol ether sulfates, alkylamide sulfates and alkylamidesulfonates, fatty acid alkylamide polyglycol ether sulfates, alkane sulfate, alkanesulfonates and hydroxyalkanesulfonates, olefinsulfonates, acyl esters of isethionates, α-sulfo fatty acid esters, alkylbenzenesulfonates, alkylphenol glycol ether sulfonates, sulfosuccinates, sulfosuccinic monoesters and diesters, fatty alcohol ether phosphates, protein-fatty acid condensation products, alkylmonoglyceride sulfates and alkylmonoglyceridesulfonates, alkylglyceride ether sulfonates, fatty acid methyltaurides, fatty acid sarcosinates, sulforicinoleates, amphoacetates or amphoglycinates, and acylglutamates. These compounds and mixtures therefore are utilized in the form of their water-soluble or water-dispersible salts, examples being the sodium, potassium, magnesium, ammonium, mono-, di-, and triethanolammonium and also analogous alkylammonium salts.

The fraction of the anionic surfactants in the preparations of the invention is preferably 1% to 30%, more preferably 5% to 25%, and with more particular preference 10% to 22% by weight, based on the completed preparations.

Suitable cationic surfactants are, for example, quaternary ammonium salts such as di-(C₁₀-C₂₄ alkyl)-dimethylammonium chloride or bromide, preferably di-(C₁₂-C₁₈ alkyl)-dimethylammonium chloride or bromide; C₁₀-C₂₄ alkyl-dimethylethylammonium chloride or bromide; C₁₀-C₂₄ alkyltrimethylammonium chloride or bromide, preferably cetyltrimethylammonium chloride or bromide and C₂₀-C₂₂ alkyl-trimethylammonium chloride or bromide; C₁₀-C₂₄ alkyl-dimethylbenzylammonium chloride or bromide, preferably C₁₂-C₁₈ alkyl-dimethylbenzylammonium chloride; N—(C₁₀-C₁₈ alkyl)pyridinium chloride or bromide, preferably N—(C₁₂-C₁₆ alkyl)pyridinium chloride or bromide; N—(C₁₀-C₁₈ alkyl)isoquinolinium chloride, bromide or monoalkyl sulfate; N—(C₁₂-C₁₈ alkylpolyoylaminoformylmethyl)pyridinium chloride; N—(C₁₂-C₁₈ alkyl)-N-methylmorpholinium chloride, bromide or monoalkyl sulfate; N—(C₁₂-C₁₈ alkyl)-N-ethylmorpholinium chloride, bromide or monoalkyl sulfate; C₁₆-C₁₈ alkyl-pentaoxethylammonium chloride; diisobutylphenoxyethoxyethyldimethylbenzylammonium chloride; salts of N,N-diethylaminoethylstearylamide and -oleylamide with hydrochloric acid, acetic acid, lactic acid, citric acid, phosphoric acid; N-acylaminoethyl-N,N-diethyl-N-methylammonium chloride, bromide or monoalkyl sulfate and N-acylaminoethyl-N,N-diethyl-N-benzylammonium chloride, bromide or monoalkyl sulfate, with acyl standing preferably for stearyl or oleyl.

The fraction of the cationic surfactants in the preparations of the invention is preferably 0.1% to 10%, more preferably 0.25 to 7%, and with more particular preference 0.5% to 5% by weight, based on the completed preparation.

Suitable nonionic surfactants which can be used as detersive substances include preferably fatty alcohol ethoxylates (alkylpolyethylene glycols); alkylphenolpolyethylene glycols; alkylmercaptanpolyethylene glycols; fatty amine ethoxylates (alkylaminopolyethylene glycols); fatty acid ethoxylates (acylpolyethylene glycols); polypropylene glycol ethoxylates (Pluronics®); fatty acid amidepolyethylene glycols; N-alkyl-, N-alkoxypolyhydroxy-fatty acid amide, more particularly fatty acid N-methylglucamides, sucrose esters; polyglycol ethers, alkylpolyglycosides, phosphoric esters (mono-, di-, and triphosphoric esters, ethoxylated and nonethoxylated).

The fraction of the nonionic surfactants in the preparations of the invention (in the case of rinse-off products, for example) is preferably in the range from 1% to 20%, more preferably from 2% to 10%, and with more particular preference from 3% to 7% by weight, based on the completed preparation.

Preferred amphosurfactants are as follows: N—(C₁₂-C₁₈ alkyl)-β-aminopropionates and N—(C₁₂-C₁₈ alkyl)-1-iminodipropionates in the form of alkali metal salts and mono-, di-, and trialkylammonium salts; N-acylaminoalkyl-N,N-dimethylacetobetaine, preferably N—(C₈-C₁₈ acyl)aminopropyl-N,N-dimethylacetobetaine; C₁₂-C₁₈ alkyl-dimethylsulfopropylbetaine; amphosurfactants based on imidazoline (trade name: Miranol®, Steinapon®), preferably the sodium salt of 1-(β-carboxymethyloxyethyl)-1-(carboxymethyl)-2-laurylimidazolinium; amine oxides, e.g., C₁₂-C₁₈ alkyldimethyl-amine oxide, fatty acid amidoalkyldimethylamine oxide.

The fraction of the amphoteric surfactants in the preparations of the invention is preferably 0.5% to 20% by weight and more preferably 1% to 10% by weight, based on the completed preparation.

Furthermore it is possible in the preparations of the invention to use foam-boosting co-surfactants from the group consisting of alkylbetaines, alkylamidobetaines, aminopropionates, aminoglycinates, imidazolinium betaines, and sulfobetaines, amine oxides and fatty acid alkanolamides or polyhydroxyamides.

The total amount of surfactants used in the preparations of the invention is preferably 1% to 70%, more preferably 10% and 40%, and with particular preference 12% to 35% by weight, based on the completed preparation.

Preparations of the invention in the form of emulsions can be produced without further emulsifier or else may comprise one or more emulsifiers. These emulsifiers may be selected from the group of the nonionic, anionic, cationic or amphoteric emulsifiers.

Suitable nonionic coemulsifers include preferably adducts of 0 to 30 mol of ethylene oxide and/or 0 to 5 mol of propylene oxide with linear fatty alcohols having 8 to 22 C atoms, with fatty acids having 12 to 22 C atoms, with alkylphenols having 8 to 15 C atoms in the alkyl group, and with sorbitan or sorbitol esters; (C₁₂-C₁₈) fatty acid monoesters and diesters of adducts of 0 to 30 mol of ethylene oxide with glycerol; glycerol monoesters and diesters and sorbitan monoesters and diesters of saturated or unsaturated fatty acids having 6 to 22 carbon atoms and, where appropriate, their ethylene oxide adducts; adducts of 15 to 60 mol of ethylene oxide with castor oil and/or hydrogenated castor oil; polyol esters and more particularly polyglycerol esters, such as polyglycerol polyricinoleate and polyglycerol poly-12-hydroxystearate, for example. Likewise suitable with preference are ethoxylated fatty amines, fatty acid amides, fatty acid alkanolamides, and mixtures of compounds from two or more of these classes of substance.

Examples of suitable ionic coemulsifiers include anionic emulsifiers, such as mono-, di- or tri-phosphoric esters, soaps (e.g., sodium stearate), fatty alcohol sulfates, but also cationic emulsifiers such as mono-, di-, and tri-alkyl quats and their polymeric derivatives.

Amphoteric emulsifiers preferentially available are alkylaminoalkyl-carboxylic acids, betaines, sulfobetaines, and imidazoline derivatives.

Additionally it is possible to use naturally occurring emulsifiers, among which beeswax, woolwax, lecithin, and sterols are preferred.

Preferred are fatty alcohol ethoxylates selected from the group of ethoxylated stearyl alcohols, cetyl alcohols, cetyl stearyl alcohols, more particularly polyethylene glycol(13) stearyl ether, polyethylene glycol(14) stearyl ether, polyethylene glycol(15) stearyl ether, polyethylene glycol(16) stearyl ether, polyethylene glycol(17) stearyl ether, polyethylene glycol(18) stearyl ether, polyethylene glycol(19) stearyl ether, polyethylene glycol(20) stearyl ether, polyethylene glycol(12) isostearyl ether, polyethylene glycol(13) isostearyl ether, polyethylene glycol(14) isostearyl ether, polyethylene glycol(15) isostearyl ether, polyethylene glycol(16) isostearyl ether, polyethylene glycol(17) isostearyl ether, polyethylene glycol(18) isostearyl ether, polyethylene glycol(19) isostearyl ether, polyethylene glycol(20) isostearyl ether, polyethylene glycol(13) cetyl ether, polyethylene glycol(14) cetyl ether, polyethylene glycol(15) cetyl ether, polyethylene glycol(16) cetyl ether, polyethylene glycol(17) cetyl ether, polyethylene glycol(18) cetyl ether, polyethylene glycol(19) cetyl ether, polyethylene glycol(20) cetyl ether, polyethylene glycol(13) isocetyl ether, polyethylene glycol(14) isocetyl ether, polyethylene glycol(15) isocetyl ether, polyethylene glycol(16) isocetyl ether, polyethylene glycol(17) isocetyl ether, polyethylene glycol(18) isocetyl ether, polyethylene glycol(19) isocetyl ether, polyethylene glycol(20) isocetyl ether, polyethylene glycol(12) oleyl ether, polyethylene glycol(13) oleyl ether, polyethylene glycol(14) oleyl ether, polyethylene glycol(15) oleyl ether, polyethylene glycol(12) lauryl ether, polyethylene glycol(12) isolauryl ether, polyethylene glycol(13) cetylstearyl ether, polyethylene glycol(14) cetylstearyl ether, polyethylene glycol(15) cetylstearyl ether, polyethylene glycol(16) cetylstearyl ether, polyethylene glycol(17) cetylstearyl ether, polyethylene glycol(18) cetylstearyl ether, polyethylene glycol(19) cetylstearyl ether, polyethylene glycol(20) cetylstearyl ether, polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, polyethylene glycol(20) oleate.

As ethoxylated alkyl ether carboxylic acid or salts thereof it is possible with advantage to use sodium laureth(11EO) carboxylate.

An advantageous alkyl ether sulfate is the sodium salt of lauryl diglycol ether sulfate; an advantageous ethoxylated cholesterol derivative is polyethylene glycol(30) cholesteryl ether. Also preferred is polyethylene glycol(25) soyasterol.

As ethoxylated triglycerides it is possible with advantage to use polyethylene glycol(60) evening primrose glyceride.

Additionally it is of advantage to select the polyethylene glycol glycerol fatty acid esters from the group of polyethylene glycol(20) glyceryl laurate, polyethylene glycol(6) glyceryl caprate/caprylate, polyethylene glycol(20) glyceryl oleate, polyethylene glycol(20) glyceryl isostearate, and polyethylene glycol(18) glyceryl oleate/cocoate.

Among the sorbitan esters particular suitability is possessed by polyethylene glycol(20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

As advantageous W/O emulsifiers it is possible to use the following: fatty alcohols having 8 to 30 carbon atoms, monoglycerol esters of saturated and/or unsaturated, branched and/or unbranched alkanecarboxylic acids with a chain length of 8 to 24, more particularly 18 to 18, C atoms, diglycerol esters of saturated and/or unsaturated, branched and/or unbranched alkanecarboxylic acids with a chain length of 8 to 24, more particularly 12 to 18, C atoms, monoglycerol ethers of saturated and/or unsaturated, branched and/or unbranched alcohols with a chain length of 8 to 24, more particularly 12 to 18, C atoms, diglycerol ethers of saturated and/or unsaturated, branched and/or unbranched alcohols with a chain length of 8 to 24, more particularly 12 to 18, C atoms, propylene glycol esters of saturated and/or unsaturated, branched and/or unbranched alkanecarboxylic acids with a chain length of 8 to 24, more particularly 12 to 18 C atoms, and also sorbitan esters of saturated and/or unsaturated, branched and/or unbranched alkanecarboxylic acids with a chain length of 8 to 24, more particularly 12 to 18, C atoms.

Especially advantageous W/O emulsifiers are glyceryl monostearate, glyceryl monoisostearate, glyceryl monomyristate, glyceryl monooleate, glyceryl monolaurate, glyceryl monocaprylate, glyceryl monocaprate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol or polyethylene glycol(2) stearyl ether.

The fraction of the emulsifier or emulsifiers in the preparations of the invention is preferably 0.1% to 20%, more preferably 0.5% to 15%, and with more particular preference 1% to 10% by weight, based on the completed preparation.

Suitable vehicles include preferably vegetable oils, natural and hydrogenated oils, waxes, fats, water, alcohols, polyols, glycerol, glycerides, liquid paraffins, liquid fatty alcohols, sterol, polyethylene glycols, and cellulose and cellulose derivatives.

Humectant substances available are preferably isopropyl palmitate, glycerol, polyethylene glycols and/or sorbitol, which are used preferably from 0.1% to 50% by weight.

Suitable preservatives include preferably phenoxyethanol, phenoxyisopropanol, 1,2-octanediol, benzyl alcohol, parabens (methyl, ethyl, propyl, butyl, and isobutyl paraben) and their salts, benzoic acid, salicylic acid, sorbic acid and its salts, piroctone olamine (Octopirox®, Clariant), silver chloride on titanium dioxide, chloroxylenol, imidazolidinyl urea, diazolidinyl urea, DMDM hydantoin, sodium hydroxymethylglycinate, 2-bromo-2-nitropropane-1,3-diol, methyldibromoglutaronitrile, iodopropynyl butylcarbamate, and methylchloroisothiazolinones and methylisothiazolinones. They are used preferably in amounts from 0.001% to 5%, more preferably from 0.01% to 3%, and with more particular preference from 0.1% to 2% by weight, based on the completed preparations.

As dyes it is possible to use the substances which are suitable and approved for cosmetic and pharmaceutical use.

As fragrance or perfume oils it is possible to use individual odorant compounds, examples being the synthetic products of the ester, ether, aldehyde, ketone, alcohol, and hydrocarbon types. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl methylphenylglycinate, allylcyclohexyl propionate, styrallyl propionate, and benzyl salicylate. The ethers include, for example, benzyl ethyl ether; the aldehydes include, for example, the linear alkanals having 8 to 18 C atoms, citral, citronellal, citronellyloxyacetaldehyde, cyclamenaldehyde, hydroxycitronellal, lilial, and bourgeonal; the ketones include, for example, the ionones, alpha-isomethylionone, and methyl cedryl ketone; the alcohols include anethol, citroneliol, eugenol, geraniol, linalool, phenylethyl alcohol, and terpineol; and the hydrocarbons include primarily the terpenes and balsams. Preference is given to using mixtures of different odorants which in unison produce a pleasant fragrance note.

Perfume oils may also comprise natural odorant mixtures, of the kind obtainable from plant or animal sources, examples being pine oil, citrus oil, jasmine oil, lily oil, rose oil, or ylang ylang oil. Essential oils of relatively low volatility, which are usually used as flavoring components, are also suitable as perfume oils, examples being sage oil, chamomile oil, oil of cloves, melissa oil, mint oil, oil of cinnamon leaves, linden blossom oil, oil of juniper berries, vetiver oil, olibanum oil, galbanum oil, and ladanum oil.

As solvents it is possible to use alcohols having 1 to 4 carbon atoms such as ethanol, propanol, isopropanol, n-butanol, isobutanol, tert-butanol or glycerol, and also alkylene glycols, more particularly propylene glycol, butylene glycol or hexylene glycol, and mixtures of the stated alcohols. Further preferred alcohols are polyethylene glycols having a relative molecular mass below 2000. Preference is given more particularly to the use of polyethylene glycol having a relative molecular mass between 200 and 600, and polyethylene glycol having a relative molecular mass between 400 and 600.

The oil-based preparations of the invention may preferably comprise the following: hydrocarbon oils with linear or branched, saturated or unsaturated C₇-C₄₀ carbon chains, for example dodecane, isododecane, cholesterol, hydrogenated polyisobutylenes, docosanes, hexadecane, isohexadecane, paraffins and isoparaffins, but also triglycerides of animal and vegetable origin, for example beef tallow, pig fat, goose grease, perhydrosqualene, lanolin, sunflower oil, maize oil, soya oil, rice oil, jojoba oil, babusscu oil, pumpkin oil, grapeseed oil, sesame oil, walnut oil, apricot oil, macadamia oil, avocado oil, sweet almond oil, lady's smock oil, castor oil, olive oil, peanut oil, rapeseed oil and coconut oil and synthetic oils, such as purcellin oil, linear and/or branched fatty alcohols and fatty acid esters, preferably Guerbet alcohols having 6 to 18, preferably 8 to 10, carbon atoms; esters of linear (C₆-C₁₃)-fatty acids with linear (C₆-C₂₀)-fatty alcohols; esters of branched (C₆-C₁₃)-carboxylic acids with linear (C₆-C₂₀)-fatty alcohols, esters of linear (C₆-C₁₈)-fatty acids with branched alcohols, in particular 2-ethylhexanol; esters of linear and/or branched fatty acids with polyhydric alcohols (such as e.g. dimerdiol or trimerdiol) and/or Guerbet alcohols; alcohol esters of C₁-C₁₀ carboxylic acids or C₂-C₃₀ dicarboxylic acids, esters, such as dioctyl adipate, diisopropyl dimer dilineolate; propylene glycol/dicaprylate or waxes, such as beeswax, paraffin wax or microcrystalline waxes, optionally in combination with hydrophilic waxes, such as, for example, cetylstearyl alcohol; fluorinated and perfluorinated oils; monoglycerides of C₁-C₃₀ carboxylic acids, diglycerides of C₁-C₃₀ carboxylic acids, triglycerides of C₁-C₃₀ carboxylic acids, for example triglycerides of caprylic/capric acids, ethylene glycol monoesters of C₁-C₃₀ carboxylic acids, ethylene glycol diesters of C₁-C₃₀ carboxylic acids, propylene glycol monoesters of C₁-C₃₀ carboxylic acids, propylene glycol diesters of C₁-C₃₀ carboxylic acids, and propoxylated and ethoxylated derivatives of the abovementioned classes of compound. The carboxylic acids can comprise linear or branched alkyl groups or aromatic groups. By way of example, mention may be made of diisopropyl sebacate, diisopropyl adipate, isopropyl myristate, isopropyl palmitate, myristyl propionate, ethylene glycol distearate, 2-ethylhexyl palmitate, isodecyl neopentanoate, di-2-ethylhexyl maleate, cetyl palmitate, myristyl myristate, stearyl stearate, cetyl stearate, behenyl behenate, dioctyl maleate, dioctyl sebacate, cetyl octanoate, diisopropyl dilinoleate, caprylic/capryl triglyceride, PEG-6 caprylic/capryl triglyceride, PEG-8 caprylic/capryl triglyceride, cetyl ricinoleate, cholesterol hydroxystearate, cholesterol isostearate, C₁-C₃₀ monoesters and polyesters of glycerol, for example glyceryl tribehenate, glyceryl stearate, glyceryl palmitate, glyceryl distearate, glyceryl dipalmitate, C₁-C₃₀ carboxylic monoesters and polyesters of sugar, for example glucose tetraoleate, glucose tetraesters of soya oil fatty acid, mannose tetraesters of soya oil fatty acid, galactose tetraesters of oleic acid, arabinose tetraesters of linoleic acid, xylose tetralinoleate, galactose pentaoleate, sorbitol tetraoleate, sorbitol hexaesters of unsaturated soya oil fatty acid, xylitol pentaoleate, sucrose tetraoleate, sucrose pentaoleate, sucrose hexaoleate, sucrose heptaoleate, sucrose oleate.

The silicone oils available are preferably dimethylpolysiloxanes and cyclomethicones, polydialkylsiloxanes R₃SiO(R₂SiO)ₙSiR₃, where R is methyl or ethyl, particularly preferably methyl, and x is a number from 2 to 500, for example the dimethicones available under the trade names Vicasil (General Electric Company), Dow Corning 200, Dow Corning 225, Dow Corning 200 (Dow Corning Corporation), trimethylsiloxysilicates [((CH₂)₃SiO)_{1/2}]ₓ[SiO₂]_{y}, where x is a number from 1 to 500 and y is a number from 1 to 500, dimethiconols R₃SiO[R₂SiO]ₓSiR₂OH and HOR₂SiO[R₂SiO]ₓSiR₂OH, where R is methyl or ethyl and x is a number up to 500, polyalkylarylsiloxanes, for example the polymethylphenylsiloxanes available under the trade names SF 1075 Methylphenyl Fluid (General Electric Company) and 556 Cosmetic Grade Phenyl Trimethicone Fluid (Dow Corning Corporation), polydiarylsiloxanes, silicone resins, cyclic silicones and amino-, fatty acid-, alcohol-, polyether-, epoxy-, fluorine- and/or alkyl-modified silicone compounds and polyether siloxane copolymers.

Of preferential suitability as antimicrobial actives are cetyltrimethylammonium chloride, cetylpyridinium chloride, benzethonium chloride, diisobutylethoxyethyldimethylbenzylammonium chloride, sodium N-laurylsarcosinate, sodium N-palmethylsarcosinate, lauroylsarcosine, N-myristoylglycine, potassium N-laurylsarcosine, trimethylammonium chloride, sodium aluminum chlorohydroxylactate, triethyl citrate, tricetylmethylammonium chloride, 2,4,4′-trichloro-2′-hydroxydiphenyl ether (triclosan), phenoxyethanol, 1,5-pentanediol, 1,6-hexanediol, 3,4,4′-trichlorocarbanilide (trichlocarban), diaminoalkylamide, L-lysine hexadecylamide for example, heavy metal citrate salts, salicylates, piroctones, more particularly piroctoneolamine, pyrithiones and their heavy metal salts, more particularly zinc pyrithione, zinc phenol sulfate, farnesol, and combinations of these active substances.

The preparations of the invention contain the antimicrobial agents preferably in amounts up to 50% by weight, more preferably in amounts from 0.01% to 10% by weight, and with more particular preference in amounts of from 0.1 to 10% by weight, based on the complete preparations.

In another preferred embodiment the preparations of the invention comprise one or more photoprotective pigments. These pigments may be selected both from organic and from inorganic photoprotective pigments.

The preferred inorganic photoprotective pigments are finely disperse or colloidally disperse metal oxides and metal salts, for example titanium oxide, zinc oxide, iron oxide, aluminum oxide, cerium oxide, zirconium oxide, silicates (talc) and barium sulfate. The particles should in this case have a mean diameter of less than 100 nm, preferably between 5 and 50 nm and particularly preferably between 15 and 30 nm, so-called ‘nanopigments’. They can have a spherical form, but it is also possible to use those particles which have an ellipsoidal shape or one which differs in another way from the spherical shape. The pigments can also be surface-treated, i.e. be present in hydrophilized or hydrophobicized form. Typical examples are coated titanium dioxides, such as, for example, titanium dioxide T 805 (Degussa) or Eusolex®T2000 (Merck). Possible hydrophobic coating agents are in this case especially silicones and in this context specifically trialkoxyoctylsilanes or dimethicones. Titanium dioxide and zinc oxide are particularly preferred.

The preferred inorganic particle substances are hydrophilic or amphiphilic. Advantageously, they can be superficially coated, in particular superficially treated to repel water. Examples of these are titanium oxide pigments coated with aluminum stearate, zinc oxide coated with dimethylpolysiloxane (dimethicone), boron nitride coated with dimethicone and titanium oxide coated with a mixture of dimethylpolysiloxane and silica gel and hydrated aluminum oxide, titanium oxide coated with octylsilanol, or spherical polyalkylsesquioxane particles.

Organic photoprotective pigments are substances which are present in crystalline form at room temperature, which are able to absorb ultraviolet rays and to emit the absorbed energy again in the form of longer wavelength radiation, e.g., heat. A distinction is made between UVA filters and UVB filters. The UVA and UVB filters can be used both individually and in mixtures.

The organic UV filters suitable according to the invention are selected from the derivatives, solid at room temperature, of dibenzoylmethane, cinnamic acid esters, diphenyl acid esters, benzophenone, camphor, p-aminobenzoic acid esters, o-aminobenzoic acid esters, salicylic acid esters, benzimidazoles, 1,3,5-triazines, monomeric and oligomeric 4,4-d iarylbutadienecarboxylic acid esters and -carboxamides, ketotricyclo-[5.2.1.0]decane, benzalmalonic acid esters, and any desired mixtures of the specified components. The organic UV filters may be oil-soluble or water-soluble. Particularly preferred oil-soluble UV filters according to the invention are (1-(4-tert-butylphenyl)-3-(4′-methoxyphenyl)propane-1,3-dione, 1-phenyl-3-(4′-isopropylphenyl)propane-1,3-dione, 3-(4′-methylbenzylidene)-D, L-camphor, 2-ethylhexyl 4-(dimethylamino)benzoate, 2-octyl 4-(dimethylamino)benzoate, amyl 4-(dimethylamino)benzoate, 2-ethylhexyl 4-methoxycinnamate, propyl 4-methoxycinnamate, isopentyl 4-methoxycinnamate, 2-ethylhexyl 2-cyano-3,3-phenylcinnamate (octocrylene), 2-ethylhexyl salicylate, 4-isopropylbenzyl salicylate, homomethyl salicylate (3,3,5-trimethyloctylhexyl salicylate), 2-hydroxy-4-methoxybenzophenone, 2-hydroxy-4-methoxy-4′-methylbenzophenone, 2,2′-dihydroxy-4-methoxybenzophenone, di-2-ethylhexyl 4-methoxybenzmalonate, 2,4,6-trianilino(p-carbo-2′-ethyl-1′-hexyloxy)-1,3,5-triazine (octyltriazone) and dioctylbutamidotriazone, and any desired mixtures of the specified components.

Preferred water-soluble UV filters are 2-phenylbenzimidazole-5-sulfonic acid and its alkali metal, alkaline earth metal, ammonium, alkylammonium, alkanolammonium and glucammonium salts, sulfonic acid derivatives of benzophenones, preferably 2-hydroxy-4-methoxybenzophenone-5-sulfonic acid and its salts, sulfonic acid derivatives of 3-benzylidenecamphor, such as, for example, 4-(2-oxo-3-bornylidenemethyl)benzenesulfonic acid and 2-methyl-5-(2-oxo-3-bornylidene)sulfonic acid and their salts.

In the preparations of the invention, the inorganic and organic photo-protective pigments are present in amounts of preferably 0.1% to 30% by weight, more preferably 1% to 20% by weight and very preferably 2% to 15% by weight, based in each case on the total weight of the preparation.

In a further preferred embodiment, the preparations according to the invention comprise one or more UV photoprotective filters.

Suitable UV filters are preferably 4-aminobenzoic acid; 3-(4′-trimethylammonium)benzylideneboran-2-one methylsulfate; 3,3,5-trimethylcyclohexyl salicylate; 2-hydroxy-4-methoxybenzophenone; 2-phenylbenzimidazole-5-sulfonic acid and its potassium, sodium and triethanolamine salts; 3,3′-(1,4-phenylenedimethine)bis(7,7-dimethyl-2-oxobicyclo-[2.2.1]-heptane-1-methanesulfonic acid and its salts; 1-(4-tert-butylphenyl)-3-(4-methoxyphenyl)propane-1,3-dione, 3-(4′-sulfo)benzylidenebornan-2-one and its salts; 2-ethylhexyl 2-cyano-3,3-diphenylacrylate; polymer of N-[2(and 4)-(2-oxoborn-3-ylidenemethyl)benzyl]acrylamide; 2-ethylhexyl 4-methoxycinnamate; ethoxylated ethyl 4-aminobenzoate; isoamyl 4-methoxycinnamate; 2,4,6-tris[p-(2-ethylhexyloxy-carbonyl)anilino]-1,3,5-triazine; 2-(2H-benzotriazol-2-yl)-4-methyl-6-(2-methyl-3-(1,3,3,3-tetramethyl-1-(trimethylsilyloxy)disiloxanyl)-propyl)phenol; 4,4′-[(6-[4-((1,1-dimethylethyl)aminocarbonyl)phenylamino]-1,3,5-triazin-2,4-yl)diimino]bis(2-ethylhexyl benzoate); 3-(4′-methylbenzylidene)-D,L-camphor; 3-benzylidenecamphor; 2-ethylhexyl salicylate; 2-ethylhexyl 4-dimethylaminobenzoate; hydroxy-4-methoxybenzophenone-5-sulfonic acid (sulisobenzonum) and the sodium salt; and/or 4-isopropylbenzyl salicylate, N,N,N-trimethyl-4-(2-oxoborn-3-ylidenemethyl)anilium methylsulfate, homosalate (INN), oxybenzone (INN), 2-phenylbenzimidazole-5-sulfonic acid and its Na, K, and triethanolamine salts, alpha-(2-oxoborn-3-ylidene)toluene-4-sulfonic acid and its salts, octyl methoxycinnamate, isopentyl 4-methoxycinnamate, isoamyl p-methoxycinnamate, 2,4,6-trianilino-(p-carbo-2′-ethylhexyl-1′-oxy)-1,3,5-triazine (octyltriazone), phenol, 2-2(2H-benzotriazol-2-yl)-4-methyl-6-(2-methyl-3-(1,3,3,3-tetramethyl-1-(trimethylsilyl)oxy)disiloxanyl)propyl (drometriazole trisiloxane)benzoic acid, 4,4-((6-(((1,1-dimethylethyl)amino)-carbonyl)phenyl)amino)-1,3,5-triazine-2,4-diyl)diimino)bis,bis(2-ethylhexyl)) ester)benzoic acid, 4,4-((6-(((1,1-dimethylethyl)amino)carbonyl)phenyl)amino)-1,3,5-triazine-2,4-diyl)-diimino)bis-, bis(2-ethylhexyl) ester) 3-(4′-methylbenzylidene)-d-1-camphor (4-methylbenzylidenecamphor), 3-benzylidenecamphor, 2-ethylhexyl salicylate (octyl salicylate), ethyl-2-hexyl 4-dimethylaminobenzoate (octyl dimethyl PABA), 2-hydroxy-4-methoxybenzophenone-5-sulfonic acid (benzophenone-5) and the Na salt, 2,2′-methylenebis-6-(2H-benzotriazol-2-yl)-4-(tetramethylbutyl)-1,1,3,3-phenol, sodium salt of 2-2′-bis(1,4-phenylene)-1H-benzimidazole-4,6-disulfonic acid, (1,3,5)-triazine-2,4-bis((4-(2-ethylhexyloxy)-2-hydroxy)phenyl)-6-(4-methoxyphenyl), 2-ethylhexyl 2-cyano-3,3-diphenyl-2-propenoate, glyceryl octanoate, di-p-methoxycinnamic acid, p-aminobenzoic acid and ester, 4-tert-butyl-4′-methoxydibenzoylmethane, 4-(2-β-glucopyranoxy)propoxy-2-hydroxybenzophenone, octyl salicylate, methyl 2,5-diisopropylcinnamate, cinoxate, dihydroxydimethoxybenzophenone, disodium salt of 2,2′-dihydroxy-4,4′-dimethoxy-5,5′-disulfobenzophenone, dihydroxybenzophenone, 1-(3,4-dimethoxyphenyl)-4,4-dimethyl-1,3-pentanedione, 2-ethylhexyl dimethoxybenzylidene dioxoimidazolidine propionate, tetrahydroxybenzophenone, terephthalylidenedicamphorsulfonic acid, 2,4,6-tris[4-2-ethylhexyloxycarbonyl)anilino]-1,3,5-triazine, methyl bis(trimethylsiloxy)silyl isopentyltrimethoxycinnamate, amyl p-dimethylaminobenzoate, 2-ethylhexyl p-dimethylaminobenzoate, isopropyl p-methoxycinnamate/diisopropyl cinnamate, 2-ethylhexyl p-methoxycinnamate, 2-hydroxy-4-methoxybenzophenone, 2-hydroxy-4-methoxybenzophenone-5-sulfonate and the trihydrate, 2-hydroxy-4-methoxybenzophenone-5-sulfonate, Na salt, and phenylbenzimidazolesulfonic acid.

The preparations of the invention comprise UV photoprotective filters in the amounts by weight of preferably 0.1% to 10%, more preferably 0.5% to 8%, and very preferably 1% to 5%, based on the completed preparations.

In a further preferred embodiment the preparations of the invention comprise one or more antioxidants.

Advantageously the antioxidants are selected from the group consisting of amino acids (e.g. glycine, histidine, tyrosine, tryptophan) and their derivatives, imidazoles (e.g. urocaninic acid) and their derivatives, peptides such as D,L-carnosine, D-carnosine, L-carnosine and their derivatives (e.g. anserine), carotenoids, carotenes (e.g. α-carotene, β-carotene, lycopene) and their derivatives, chlorogenic acid and its derivatives, lipoic acid and its derivatives (e.g. dihydrolipoic acid), aurothioglucose, propylthiouracil and other thiols (e.g. thioredoxin, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters) and their salts, dilauryl thiodipropionate, distearyl thiodipropionate, thiodipropionic acid and its derivatives (esters, ethers, peptides, lipids, nucleotides, nucleosides and salts) and sulfoximine compounds (e.g. buthionine sulfoximine, homocysteine sulfoximine, buthionine sulfone, penta-, hexa-, heptathionine sulfoximine) in very low tolerable doses, furthermore (metal) chelators (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and their derivatives, unsaturated fatty acids and their derivatives (e.g. γ-linolenic acid, linoleic acid, oleic acid), folic acid and its derivatives, ubiquinone and ubiquinol and their derivatives, vitamin C and derivatives (e.g. ascorbylpalmitate, Mg ascorbylphosphate, ascorbylacetate), tocopherols and derivatives (e.g. vitamin E acetate), vitamin A and derivatives (vitamin A palmitate) and coniferyl benzoate of benzoin resin, rutic acid and its derivatives, α-glycosylrutin, ferulic acid, furfurylideneglucitol, carnosine, butylhydroxytoluene, butylhydroxyanisole, nordyhydroguaiac resin acid, nordihydroguaiaretic acid, trihydroxybutyrophenone, uric acid and its derivatives, mannose and its derivatives, zinc and its derivatives (e.g. ZnO, ZnSO₄), selenium and its derivatives (e.g. selenomethionine), stilbenes and their derivatives (e.g. stilbene oxide, trans-stilbene oxide), superoxide dismutase and the derivatives suitable according to the invention (salts, esters, ethers, sugars, nucleotides, nucleosides, peptides and lipids) of the substances mentioned.

Water-soluble antioxidants can be used particularly advantageously within the meaning of the present invention.

The antioxidants are able to protect the skin and the hair from oxidative stress. Preferred antioxidants here are vitamin E and its derivatives and vitamin A and its derivatives.

The amount of the one or more antioxidants in the preparations according to the invention is preferably 0.001% to 30% by weight, more preferably 0.05% to 20% by weight, and very preferably 1% to 10% by weight, based on the total weight of the preparations.

If vitamin E and/or its derivatives is/are the antioxidant(s), it is advantageous to choose its/their respective concentrations from the range from 0.001% to 10% by weight, based on the total weight of the preparation.

If vitamin A, or vitamin A derivatives, or carotenes or their derivatives is/are the antioxidant(s), it is advantageous to choose its/their respective concentrations from the range from 0.001% to 10% by weight, based on the total weight of the preparation.

In one particularly preferred embodiment of the invention the preparations comprise antioxidants selected from superoxide dismutase, tocopherol (vitamin E), and ascorbic acid (vitamin C).

As acids or alkalis for pH adjustment it is preferred to use mineral acids, HCl for example, inorganic bases, NaOH and KOH for example, and organic acids, preferably citric acid.

The preparations of the invention are adjusted preferably to a pH in the 2 to 12 range, and more preferably 3 to 8.

The preparations of the invention may take the form of a lotion, paste, cream, gel or any other typical application form.

The preparations of the invention are suitable advantageously as abrasives. The present invention hence also provides for the use of the preparations of the invention as abrasives.

The preparations of the invention can be prepared using the nanocorundum described above.

Very advantageously for the formulator the nanocorundum particles or modified nanocorundum particles used in accordance with the invention may be made available in the form of aqueous dispersion concentrates. These concentrates contain nanocorundum particles in amounts of preferably 10% to 70%, more preferably 20% to 60%, and with more particular preference 40% to 55%, by weight, and can be incorporated into the preparation or formulation in a simple way without direct handling of a dust-forming powder.

The present invention hence also provides a process for producing a preparation of the invention by introducing the nanocrystalline corundum in the form of an aqueous dispersion concentrate, preferably containing 10% to 70%, more preferably 20% to 60%, and with more particular preference 40% to 55%, by weight, of the nanocrystalline corundum, into the preparation.

The aqueous dispersion concentrates preferably comprise one or more stabilizers and/or dispersants.

In one preferred embodiment of the invention the aqueous dispersion concentrates comprise one or more stabilizers, preferably HCl and/or citric acid.

In another preferred embodiment of the invention the aqueous dispersion concentrates comprise one or more dispersants or dispersing agents, preferably selected from polyacrylates, polyvinyl alcohols, surfactants, and polyethylene glycols, more preferably polyacrylates.

The aqueous dispersion concentrates contain the one or more stabilizers and/or dispersants preferably in the amounts of 0.05% to 10% by weight.

The examples and applications below are intended to elucidate the invention but without restricting it to them (all percentages are by weight).

### PREPARATION EXAMPLE 1

### Preparation of a Nanocorundum Dispersion

A 50% strength by weight aqueous solution of aluminum chlorohydrate was admixed with 2% by weight of crystallization nuclei of a suspension of ultrafine corundum. When the solution had been homogenized by stirring, drying took place in a rotary evaporator. The solid aluminum chlorohydrate was comminuted in a mortar producing a coarse powder.

The powder was calcined in a muffle furnace at 1050° C. The contact time in the hot zone was not more than 5 minutes. This gave a white powder whose particle size distribution corresponded to the feed material.

X-ray structural analysis shows that this powder is pure-phase α-aluminum oxide.

The images of the SEM (scanning electron microscope) micrograph taken showed crystallites in the 10-100 nm range. The residual chlorine content was just a few ppm.

150 g of this corundum powder was suspended in 150 g of water. The suspension was passed to a vertical agitator ball mill from Netzsch (model PE 075). The grinding beads used were of zirconium oxide (stabilized with yttrium) and had a size of 0.3-0.5 mm.

The particle size distribution of the feed material was between 20-100 μm. After each of one, two, three, and four hours, the mill was halted and a sample taken. At these times, in addition, the pH was checked. The pH, which increases as deagglomeration progresses, was maintained at a level of 5 by addition of dilute hydrochloric acid. The samples taken hourly were characterized using a Horiba particle sizer.

It was found that after 4 hours the primary crystallites had undergone only slight agglomeration. The coarse primary particles (20-100 μm) were almost completely destroyed after just 1 hour, and so after this time a d50 of approximately 270 nm was found. The d50 after 4 hours was <108 nm.

### PREPARATION EXAMPLE 2

### Preparation of an Aqueous Nanoruby Dispersion

Preparation took place as in preparation example 1. In deviation from that example the 50% strength by weight aqueous aluminum chlorohydrate solution was admixed with 0.5% by weight of Cr(III) chloride, based on the solids content. This gave a pink suspension of nanoruby.

### PREPARATION EXAMPLE 3

### Preparation of an Aqueous Nanosapphire Dispersion

Preparation took place as in preparation example 1. In deviation from that example the 50% strength by weight aqueous aluminum chlorohydrate solution was admixed with 0.1% by weight of iron(II) sulfate, based on the solids content. This gave a pale bluish suspension of nanosapphire.

### PREPARATION EXAMPLE 4

### Preparation of a Hydrophobically Modified Nanocorundum

150 g of corundum powder with a grain size in the 10-50 μm range, consisting of crystallites <100 nm (prepared as in preparation example 1), were suspended in 110 g of isopropanol. The suspension was admixed with 40 g of trimethoxyoctylsilane and supplied to a vertical agitator ball mill from Netzsch (model PE 075). The grinding beads used were of zirconium oxide (stabilized with yttrium) and had a size of 0.3-0.5 mm. After three hours the suspension was separated from the grinding beads and boiled under reflux for a further 4 hours. Subsequently the solvent was removed by distillation and the wet residue that remained was dried in a drying oven at 110° C. for a further 20 hours. The images of the SEM (scanning electron microscope) micrograph taken showed the presence of crystallites in the 10-100 nm range. The residue was suspended in water (50% by weight suspension) and deagglomerated by ultrasonication, giving hydrophobically modified particles in the 10-100 nm range.

### PREPARATION EXAMPLE 5

A 50% strength aqueous solution of aluminum chlorohydrate was admixed with magnesium chloride such that, following calcining, the ratio of aluminum oxide to magnesium oxide was 99.5%:0.5%. In addition the solution was admixed with 2% crystallization nuclei of a suspension of ultrafine corundum. When the solution had been homogenized by stirring, drying took place in a rotary evaporator. The solid aluminum chlorohydrate/magnesium chloride mixture was comminuted in a mortar producing a coarse powder.

The powder was calcined in a rotary tube furnace at 1050° C. The contact time in the hot zone was not more than 5 minutes. This gave a white powder whose particle size distribution corresponded to the feed material.

X-ray structural analysis shows that the powder is predominantly α-aluminum oxide. The images of the SEM (scanning electron microscope) micrograph taken showed crystallites in the 10-80 nm range (estimation from SEM micrograph), present in the form of agglomerates. The residual chlorine content was just a few ppm.

In a further step, 40 g of this magnesium oxide-doped corundum powder were suspended in 160 g of water. The suspension was deagglomerated in a vertical agitator ball mill from Netzsch (model PE 075). The grinding beads used were of zirconium oxide (stabilized with yttrium) and had a size of 0.3 mm. The pH of the suspension was checked every 30 minutes and maintained at a level of 4-4.5 by addition of dilute nitric acid. After 6 hours the suspension was separated from the grinding beads and its particle size distribution was characterized using an analytical disk centrifuge from Brookhaven. The figures found were a d90 of 54 nm, a d50 of 42 nm, and a d10 of 22 nm. The nanosuspension comprising the mixed oxides is therefore significantly finer than comparable suspensions comprising pure α-aluminum oxide.

### PREPARATION EXAMPLE 6

A 50% strength aqueous solution of aluminum chlorohydrate was admixed with calcium chloride such that, following calcining, the ratio of aluminum oxide to calcium oxide was 99.5%:0.5%. In addition the solution was admixed with 2% crystallization nuclei of a suspension of ultrafine corundum. When the solution had been homogenized by stirring, drying took place in a rotary evaporator. The solid aluminum chlorohydrate/calcium chloride mixture was comminuted in a mortar producing a coarse powder.

The powder was calcined in a rotary tube furnace at 1050° C. The contact time in the hot zone was not more than 5 minutes. This gave a white powder whose particle size distribution corresponded to the feed material.

X-ray structural analysis shows that the powder is predominantly α-aluminum oxide.

The images of the SEM (scanning electron microscope) micrograph taken showed crystallites in the 10-80 nm range (estimation from SEM micrograph), present in the form of agglomerates. The residual chlorine content was just a few ppm.

In a further step, 40 g of this calcium oxide-doped corundum powder were suspended in 160 g of water. The suspension was deagglomerated in a vertical agitator ball mill from Netzsch (model PE 075). The grinding beads used were of zirconium oxide (stabilized with yttrium) and had a size of 0.3 mm. The pH of the suspension was checked every 30 minutes and maintained at a level of 4-4.5 by addition of dilute nitric acid. After 6 hours the suspension was separated from the grinding beads and its particle size distribution was characterized using an analytical disk centrifuge from Brookhaven. The figures found were a d90 of 77 nm, a d50 of 55 nm, and a d10 of 25 nm. The nanosuspension comprising the mixed oxides is therefore significantly finer than comparable suspensions comprising pure α-aluminum oxide.

### PREPARATION EXAMPLE 7

A 50% strength aqueous solution of aluminum chlorohydrate was admixed with magnesium chloride such that, following calcining, the ratio of aluminum oxide to magnesium oxide was 99.5%:0.5%. In addition the solution was admixed with 2% crystallization nuclei of a suspension of ultrafine corundum. When the solution had been homogenized by stirring, drying took place in a rotary evaporator. The solid aluminum chlorohydrate/magnesium chloride mixture was comminuted in a mortar producing a coarse powder.

The powder was calcined in a rotary tube furnace at 1050° C. The contact time in the hot zone was not more than 5 minutes. This gave a white powder whose particle size distribution corresponded to the feed material.

X-ray structural analysis shows that the powder is predominantly α-aluminum oxide.

The images of the SEM (scanning electron microscope) micrograph taken showed crystallites in the 10-80 nm range (estimation from SEM micrograph), present in the form of agglomerates. The residual chlorine content was just a few ppm.

In a further step, 40 g of this magnesium oxide-doped corundum powder were suspended in 160 g of isopropanol. The suspension was admixed with 40 g of trimethoxyoctylsilane and supplied to a vertical agitator ball mill from Netzsch (model PE 075). The grinding beads used were of zirconium oxide (stabilized with yttrium) and had a size of 0.3 mm. After 3 hours the suspension was separated from the grinding beads and boiled under reflux for a further 4 h. Subsequently the solvent was removed by distillation and the wet residue that remained was dried in a drying oven at 110° C. for a further 20 h.

### PREPARATION EXAMPLE 8

40 g of the oxide mixture (MgO-doped corundum) from preparation example 7 were suspended in 160 g of methanol and deagglomerated in a vertical agitator ball mill from Netzsch (model PE 075). After 3 h the suspension was separated from the beads and transferred to a round-bottomed flask with reflux condenser. The suspension was admixed with 40 g of trimethoxyoctylsilane and heated under reflux to 2 h. Following removal of the solvent the coated oxide mixture was isolated and was dried in a drying oven at 110° C. for a further 20 h. The resulting product is identical to the sample from preparation example 7.

### PREPARATION EXAMPLE 9

40 g of the oxide mixture (MgO-doped corundum) from preparation example 7 were suspended in 160 g of acetone and deagglomerated in a vertical agitator ball mill from Netzsch (model PE 075). After 2 h 20 g of aminopropyltrimethoxysilane (Dynasilan Ammo; Degussa) were added and the suspension was deagglomerated in the agitator ball mill for a further 2 h. Subsequently the suspension was separated from the beads and transferred to a round-bottomed flask with reflux condenser. For a further 2 h it was heated under reflux, before the solvent was removed by distillation.

### PREPARATION EXAMPLE 10

40 g of the oxide mixture (MgO-doped corundum) from preparation example 7 were suspended in 160 g of n-butanol and deagglomerated in a vertical agitator ball mill from Netzsch (model PE 075). After 2 h a mixture of 5 g of aminopropyltrimethoxysilane (Dynasilan Glymo; Degussa) and 15 g of octyltriethoxysilane was added and the suspension was deagglomerated in the agitator ball mill for a further 2 h. The suspension remains stable for weeks without signs of sedimentation of the coated mixed oxide.

### INVENTIVE EXAMPLE 1

Aqueous peel gel with nanocorundum dispersion

| | | % by weight |
|---|---|---|
| A | Aristoflex ® AVC | 1.00 |
| B | water | ad 100 |
| C | nanocorundum dispersion from preparation example 1 | 3.00 |

### Preparation:

I dissolve A in B with stirring and continue stirring until a clear gel is formed
II incorporate C into I by stirring

Addition of the nanocorundum dispersion was followed by 5 minutes of further stirring. This gave a homogeneous, white, glossy gel. When this gel is rubbed into the skin and subsequently rinsed, a significant improvement in skin sensation is obtained (smooth, tender skin as a result of the abrasive action of the nanoparticles).

### COMPARATIVE EXAMPLE 1

Aqueous peel gel with Aeroxide ® Alu C (Degussa)

| | | % by weight |
|---|---|---|
| A | Aristoflex ® AVC | 1.00 |
| B | water | ad 100 |
| C | Aeroxide ® Alu C (Degussa) | 3.00 |

### Preparation:

I. dissolve A in B with stirring and continue stirring until a clear gel is formed
II. incorporate C into I by stirring

Addition of the Aeroxide® Alu C (highly disperse pyrogenic aluminum oxide, primary particle size 13 nm) was followed by 30 minutes of further stirring. This gave a glassy gel infiltrated with undispersed aluminum oxide. The aluminum oxide powder formed copious amounts of dust during its incorporation. When this gel is rubbed into the skin and subsequently rinsed, an abrasive action is obtained, but with a slightly greasy skin sensation.

### INVENTIVE EXAMPLE 2

Peel cream gel with nanocorundum dispersion

| | | % by weight |
|---|---|---|
| A | liquid paraffin | 5.00 |
| | isopropylpalmitate | 3.00 |
| B | Aristoflex ® AVC | 1.30 |
| C | glycerol | 3.00 |
| | water | ad 100.00 |
| D | nanocorundum dispersion from preparation example 1 | 3.00 |

### Preparation:

I incorporate B into A with stirring
II incorporate C into I using a finger stirrer and continue stirring until a homogeneous cream gel is obtained
III incorporate D into II with stirring

Addition of the nanocorundum dispersion was followed by about 5 minutes of further stirring. This gave a homogeneous, white, glossy cream gel. When this gel is rubbed into the skin and subsequently rinsed, a significant improvement in skin sensation is obtained (smooth, tender skin as a result of the abrasive action of the nanoparticles).

### COMPARATIVE EXAMPLE 2

Peel cream gel with Aeroxide ® Alu C (Degussa)

| | | % by weight |
|---|---|---|
| A | liquid paraffin | 5.00 |
| | isopropylpalmitate | 3.00 |
| B | Aristoflex ® AVC | 1.30 |
| C | glycerol | 3.00 |
| | water | ad 100.00 |
| D | Aeroxide ® Alu C (Degussa) | 1.50 |

### Preparation:

I incorporate B into A with stirring
II incorporate C into I using a finger stirrer and continue stirring until a homogeneous cream gel is obtained
III incorporate D into II with stirring

Addition of the Aeroxide® Alu C (highly disperse pyrogenic aluminum oxide, primary particle size 13 nm) was followed by 1 hour of further stirring. This gave a white, highly viscous cream gel of dull appearance. The aluminum oxide powder formed copious amounts of dust during its incorporation. When this gel is rubbed into the skin and subsequently rinsed, an abrasive effect is obtained.

As shown in inventive examples 1 and 2 and comparative examples 1 and 2, in comparison with commercially customary, highly disperse aluminum oxide grades (Aeroxide® Alu C (Degussa)), the nanocorundum dispersions of the invention can be incorporated into cosmetic formulations more easily, quickly, homogeneously, and dustlessly. The skin sensation obtained after rubbing in was assessed by a sensory assessment panel as being smoother/milder and less greasy.

### INVENTIVE EXAMPLE 3

Aqueous peel gel with nanoruby dispersion

| | | % by weight |
|---|---|---|
| A | Aristoflex ® AVC | 1.00 |
| B | water | ad 100 |
| C | nanoruby dispersion from preparation example 2 | 2.00 |

### Preparation:

I dissolve A in B with stirring and continue stirring until a clear gel is formed
II incorporate C into I with stirring

### INVENTIVE EXAMPLE 4

Aqueous peel gel with nanosapphire dispersion

| | | % by weight |
|---|---|---|
| A | Aristoflex ® AVC | 1.00 |
| B | water | ad 100 |
| C | nanosapphire dispersion from preparation example 3 | 4.00 |

### Preparation:

I dissolve A in B with stirring and continue stirring until a clear gel is formed
II incorporate C into I with stirring

### INVENTIVE EXAMPLE 5

Peel cream gel with nanosapphire dispersion

| | | % by weight |
|---|---|---|
| A | liquid paraffin | 5.00 |
| | isopropylpalmitate | 3.00 |
| B | Aristoflex ® AVC | 1.30 |
| C | glycerol | 3.00 |
| | water | ad 100.00 |
| D | nanosapphire dispersion from preparation example 3 | 6.00 |

### Preparation:

I incorporate B into A with stirring
II incorporate C into I using a finger stirrer and continue stirring until a homogeneous cream gel is obtained
III incorporate D into II with stirring

### INVENTIVE EXAMPLE 6

Peel cream gel with hydrophobized nanocorundum dispersion

| | | % by weight |
|---|---|---|
| A | liquid paraffin | 5.00 |
| | isopropylpalmitate | 3.00 |
| B | Aristoflex ® AVC | 1.30 |
| C | glycerol | 3.00 |
| | water | ad 100.00 |
| D | nanocorundum dispersion from preparation example 4 | 5.00 |

### Preparation:

I incorporate B into A with stirring
II incorporate C into I using a finger stirrer and continue stirring until a homogeneous cream gel is obtained
III incorporate D into II with stirring

### INVENTIVE EXAMPLE 7

Refreshing mask

| | | % by weight |
|---|---|---|
| A | Velsan ® P8-3 | 2.00 |
| | stearyl alcohol | 1.00 |
| | cetyl alcohol | 3.00 |
| | Myritol ® 318 | 5.00 |
| | Crodamol ® AB | 4.00 |
| | SilCare ® Silicone 41M15 | 1.50 |
| | Fugogel ® 1000PP | 0.50 |
| | Dry Flo ® PC | 0.50 |
| | Eutanol ® G | 2.00 |
| | jojoba oil | 2.00 |
| | SilCare Silicone ® 15M50 | 1.00 |
| B | Aristoflex ® AVC | 1.00 |
| C | water | ad 100 |
| | allantoin | 0.20 |
| | glycerol | 2.00 |
| | magnesium gluconate | 0.20 |
| | Polyglykol 1500 S | 3.00 |
| | titanium dioxide | 2.00 |
| | kaolin | 4.00 |
| | Sorbitol ® F Liq | 0.50 |
| D | ethanol | 5.00 |
| | tocopheryl acetate | 0.50 |
| | panthenol | 1.00 |
| E | Nipaguard ® PDU | q.s. |
| F | nanocorundum dispersion from preparation example 1 | 2.00 |

### Preparation:

I mix components A and heat to 70° C.
II add B to I
III add C to II at 70° C. with vigorous stirring
IV add D to III at about 35° C.
V after 5 minutes' stirring, add E to IV
VI add F with stirring

### INVENTIVE EXAMPLE 8

Cleansing lotion

| | | % by weight |
|---|---|---|
| A | Hostacerin ® DGI | 2.00 |
| | cetyl alcohol | 1.50 |
| | liquid paraffin | 15.00 |
| | Cetiol ® SN | 8.00 |
| | Solulan 98 | 2.00 |
| B | Aristoflex ® AVC | 0.60 |
| C | Hostapon ® KCG | 0.60 |
| | water | ad 100 |
| D | hydrophobic nanocorundum from preparation | 6.00 |
| | example 4 | |

### Preparation:

I melt the components of A at 60° C., then add B
II heat C to about 60° C.
III incorporate II into I with a finger stirrer
IV stir until cold
V add D at about 35° C.

The compositions set out explicitly for inventive examples 1 to 8 were prepared analogously also with all of the other nanocrystalline products obtained according to preparation examples 1 to 10. As in preparation example 4, the hydrophobic nanocorundums of preparation examples 7-10, obtained as solids, were suspended in water and deagglomerated.

Chemical identification of the commercial products employed

| | |
|---|---|
| Aristoflex ® AVC | ammoniumacryloyldimethyltaurate/VP |
| | copolymer |
| Cetiol ® SN | cetearylisononanoate |
| Crodamol ® AB | C₁₂₋₁₅ alkylbenzoate |
| Dry Flo ® PC | aluminum starch octenylsuccinate |
| Eutanol ® G | octyldodecanol |
| Fugogel ® 1000PP | biosaccharide gum-1 |
| Hostacerin ® DGI | polyglyceryl-2-sesquiisostearate |
| Hostapon ® KCG | sodium cocoylglutamate |
| Myritol ® 318 | caprylic/capric triglyceride |
| Nipaguard ® PDU | propylene glycol/diazolidinyl urea/methyl- |
| | paraben/propylparaben |
| Polyglykol 1500 S | PEG-32 |
| SilCare ® Silicone 15M50 | phenyltrimethicone |
| SilCare ® Silicone 41M15 | caprylylmethicone |
| Solulan 98 | polysorbate 80 and cetylacetate and |
| | acetylated lanolin alcohol |
| Sorbitol ® F Liq | sorbitol |
| Velsan ® P8-3 | isopropyl C12-15-pareth-9 carboxylate |

1. A cosmetic or dermatological preparation comprising nanocrystalline corundum having particle sizes from 10 to 100 nm, and d50 values from 30 to 60 nm.

2. The cosmetic or dermatological preparation as claimed in claim 1, comprising nanocrystalline corundum having particle sizes from 10 to 100 nm, and d50 values from 30 to 60 nm, wherein the nanocrystalline corundum is produced by a process comprising the steps of:
a) mixing an aqueous solution of aluminum chlorohydrate of the formula Al₂(OH)ₓCl_{y}, in which x is a number from 2.5 to 5.5 and y is a number from 3.5 to 0.5 and the sum of x and y is always 6, with a solid, pulverulent crystallization nuclei having an average particle size of less than 0.1 μm, to produce an aqueous composition,
b) subsequently drying the aqueous composition produced by step a),
c) subsequently thermally treating the product produced by step b) at 500 to 1100° C. to produce agglomerates, and
d) comminuting the agglomerates from step c) by wet or dry grinding.

3. The preparation as claimed in claim 1, wherein the nanocrystalline corundum is unmodified.

4. The preparation as claimed in claim 2, wherein the nanocrystalline corundum is modified.

5. The preparation as claimed in claim 4, wherein the modified nanocrystalline corundum is a nanocrystalline corundum which is produced by a process wherein the aqueous composition produced by step a) further comprises at least one oxide and/or one oxide former.

6. The preparation as claimed in claim 5, wherein the at least one oxide is selected from the group consisting of: calcium oxide, magnesium oxide, chromium(III) oxide, Fe(II) oxide and Ti(IV) oxide.

7. The preparation as claimed in claim 5, wherein the at least one oxide former is selected from the group consisting of: a chloride, oxychloride, carbonate, nitrate, sulfate and hydroxychloride, of the elements of the main groups II to V and the transition groups of the Periodic Table.

8. The preparation as claimed in claim 5 wherein the at least one oxide former is selected from the group consisting of: a chloride, oxychloride, carbonate, nitrate, sulfate and hydroxychloride, of the elements Ca, Mg, Y, Ti, Zr, Cr, Fe, Co, and Si.

9. The preparation as claimed in claim 4, wherein the modified nanocrystalline corundum is doped with calcium oxide and/or with magnesium oxide.

10. The preparation as claimed in claim 4, wherein the modified nanocrystalline corundum contains 0.01% to 5% by weight of an additional oxide.

11. The preparation as claimed in claim 4, wherein the modified corundum is a nanocrystalline corundum which is surface-modified.

12. The preparation as claimed in claim 11, wherein the nanocrystalline corundum has been surface modified using at least one silane.

13. The preparation as claimed in claim 12, wherein the surface modification is performed during the deagglomeration of step d) in the presence of the at least one silane.

14. The preparation as claimed in claim 12, wherein the nanoparticles resulting from step d) are subsequently surface modified by treating with the at least one silane.

15. The preparation as claimed in claim 4, wherein modified nanocrystalline corundum has particle sizes from 20 to 80 nm.

16. The preparation as claimed in claim 1, further comprising at least one water-soluble or water-swellable crosslinked or noncrosslinked homopolymer or copolymer based on acryloyldimethyltaurine or a salt thereof and/or at least one hydrophobically modified copolymer based on acryloyidimethyltaurine or a salt thereof.

17. An abrasive, comprising the cosmetic or dermatological preparation as claimed in claim 1.

18. A process for producing a cosmetic or dermatological preparation as claimed in claim 1, wherein the nanocrystalline corundum is introduced in the form of an aqueous dispersion concentrate, into the preparation.

19. The process as claimed in claim 18, wherein the aqueous dispersion concentrate further comprises at least one stabilizer and/or dispersant.

20. The process as claimed in claim 19, wherein the aqueous dispersion concentrate comprises HCl and/or citric acid as a stabilizer.

21. The process as claimed in claim 19, wherein the aqueous dispersion concentrate further comprises at least one dispersant.

22. The process as claimed in claim 21, wherein the aqueous dispersion concentrate, contains from 0.05% to 10% by weight of the at least one stabilizer and/or dispersant.
